(19) United States
(12) Patent Application Publication
(10) Pub. No.: US 2005/0202506 A1
(43) Pub. Date: Sep. 15, 2005

(54) Methods for identifying and producing specific amino acid dependent antibodies and uses thereof

(76) Inventor: Thomas L. Cantor, El Cajon, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/799,476

(22) Filed: Mar. 11, 2004

(51) Int. Cl. G01N 33/53; C07K 16/18

(52) U.S. Cl. 435/7.1; 530/387.1

(57) ABSTRACT

The present invention relates to methods for identifying and producing amino acid dependant antibodies to specific ligands and uses thereof.

### TECHNICAL FIELD

The present invention relates to identifying and producing specific amino acid dependant antibodies and uses thereof.

### BACKGROUND ART

Since the time immunoassays were introduced in the 1960's, it has been recognized that the specificity and sensitivity of the immunoassay, from both an analytical and clinical viewpoint, depended on the areas of the analyte to which the immunoassay antibodies bound (e.g., epitopes). In many cases it was recognized that analytes that were not of interest shared a common epitope with the analyte of interest. A common approach for excluding measurement of these undesired analytes was to search through a large array of antibody candidates and select an immunoassay antibody that bound to an epitope that was not commonly shared with undesired analytes. For example, human gonadotropins including HCG (human chorionic gonadotropin), LH (leutinizing hormone) and FSH (follicle stimulating hormone) each possess both alpha and beta subunits. The alpha chains of all three of these hormones have epitopes with common homologies. Therefore, in order to specifically measure one gonadotropin while excluding the measurement of the other gonadotropins, antibodies to the beta subunit were sought.

In leading to the present disclosure it was recognized that receptors are similar to antibodies in that they will generally attach to certain epitopes of their activators. Moreover, these activators are often the analytes of interest in specific immunoassays. In seeking to develop an immunoassay that is specific for the aspect of biological activity of a receptor activating analyte it was realized that it is important to select for immunoassay antibodies having the same epitope specificity as those of the receptor.

Accordingly, there exists a need in the art for an alternative to the approach of haphazard searching for an antibody with dependence on one or a limited few amino acids. The present invention addresses this and other related needs in the art.

### DISCLOSURE OF THE INVENTION

In one aspect, the present invention is directed to a method for identifying a specific amino acid residue dependent antibody to a target protein or peptide, which method comprises: providing an antibody that binds to a target protein or peptide containing a specific amino acid residue; contacting said antibody with a negative screening protein or peptide comprising said target protein or peptide wherein said specific amino acid residue is lacking or unavailable for binding with said antibody; and assessing binding between said antibody and said target protein or peptide and assessing binding between said antibody and said negative screening protein or peptide, thereby identifying an antibody that binds to said target protein or peptide, but fails to bind to said negative screening protein or peptide as a specific amino acid residue dependent antibody. On occasion, the antibody that binds to a target protein or peptide containing a specific amino acid residue is contained in an antibody mixture and the method further comprises recovering the identified amino acid residue dependent antibody from the antibody mixture.

In one embodiment, the antibody that binds to a target protein or peptide is provided by immunizing a mammal with an immunizing protein or peptide comprising said target protein or peptide or an immunizing nucleic acid encoding a protein or peptide comprising said target protein or peptide. Frequently, the antibody that binds to a target protein or peptide is provided by identifying an antibody that is known to bind to the target protein or peptide. Further, the antibody that binds to a target protein or peptide is frequently a polyclonal antibody or polyclonal antiserum. In a related embodiment, the antibody that binds to a target protein or peptide is a monoclonal antibody or a plurality of monoclonal antibodies. In another embodiment, the antibody that binds to a target protein or peptide, specifically binds to a target protein or peptide.

In a further embodiment, the immunizing protein or peptide comprises more amino acid residue(s) than the target protein or peptide and the immunizing nucleic acid encodes a protein or peptide that comprises more amino acid residue(s) than the target protein or peptide. Frequently, the immunizing protein or peptide is the target protein or peptide and the immunizing nucleic acid encodes the target protein or peptide.

In another embodiment, the target protein or peptide is a marker of a biological pathway, a group of cellular structures with identical or similar biological function, a stage of cell cycle, a cell type, a tissue type, an organ type, a developmental stage, a disease or disorder type or stage, or a drug or other treatment monitoring. Frequently, the target protein or peptide is a clinical marker and/or a marker of parathyroid gland disease status, renal bone disease, osteoporosis, bone turnover status, the extent of partial or complete parathyroid gland removal. In addition, on occasion the target protein or peptide does not comprise a non-proteineous or non-peptidyl moiety.

In a related embodiment, the target protein or peptide is selected from the group consisting of parathyroid hormone (PTH), gastric inhibitory polypeptide (GIP), glucagon-like peptide (GLP), creatine kinase (CK), prostate specific antigen (PSA) and human chorionic gonadotropin (HCG), or a fragment thereof. Frequently, the target protein or peptide is PTH, or a fragment thereof and is used to identify an antibody that depends on one or more amino acid residues of PTH, e.g., the first or first two amino acid residues of PTH. In a related aspect, the target protein or peptide is used to identify an antibody that depends on the last amino acid residue of PTH.

In a further less occasional embodiment, the target protein or peptide may be a hormone and/or comprises a non-proteineous or non-peptidyl moiety. And, frequently, the non-proteineous or non-peptidyl moiety may be selected from the group consisting of an oligonucleotide, a nucleic acid, a vitamin, an oligosaccharide, a carbohydrate, a lipid, a small molecule and a complex or combination thereof. In such cases, the antibody is an antibody that is specific for one or more specific nucleotides or monomeric components of the non-proteineous or non-peptidyl moiety.

In another embodiment, the negative screening protein or peptide lacks one, two, three, four, five, six, seven, eight, nine, ten or more than ten amino acid residues from the target protein or peptide. Furthermore, the binding of the identified amino acid residue dependent antibody may depend on one specific amino acid residue of the target protein or peptide. Frequently, the binding of the identified amino acid residue dependent antibody depends on two or more specific amino acid residues of the target protein or peptide.

In a further embodiment, the binding (or lack thereof) between the antibody and the negative screening protein or peptide is assessed by a sandwich, competitive or inhibition assay format. In a related aspect, the binding (or lack thereof) between the antibody and the negative screening protein or peptide may be assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, u-capture assay, inhibition assay and avidity assay.

In another embodiment, the methods described above further comprise attaching the identified specific amino acid residue dependent antibody to a surface of a solid phase device suitable for testing for a target protein or peptide. And, the solid phase device may be selected from the group consisting of a microtiter plate, a glass slide, a nitrocellulose membrane, a latex bead, a cell, a test tube, a plastic bead, a colloidal gold particle, a colored particle, a magnetic bead and a quantum dot.

In one embodiment, the methods described above further comprise attaching the identified specific amino acid residue dependent antibody to a label. And, wherein the label may be selected from the group consisting of a chemical, an enzymatic, a radioactive, a fluorescent, a fluorescence-quenching, a luminescent and a fluorescence resonance energy transfer (FRET) label.

In another aspect, a specific amino acid residue dependent antibody suitable for testing for a target protein or peptide is provided, which specific amino acid residue dependent antibody is produced by the methods described above.

In a further aspect, devices suitable for testing for a target protein or peptide, which device is produced by the methods described above are provided.

In another aspect, a method is provided for producing a specific amino acid residue dependent antibody to a target protein or peptide, which method comprises: providing an antibody mixture wherein at least one of said antibodies in said mixture binds to a target protein or peptide containing a specific amino acid residue; contacting said antibody mixture provided in step a) with a negative screening protein or peptide comprising said target protein or peptide wherein said specific amino acid residue is lacking or unavailable for binding with said antibody; removing from said mixture an undesired antibody that binds to said negative screening protein or peptide; and recovering from said mixture a desired antibody that binds to said target protein or peptide but fails to bind to said negative screening protein or peptide as a specific amino acid residue dependent antibody.

In one embodiment, the negative screening protein or peptide is attached to a solid phase. And, frequently the solid phase is selected from the group consisting of an agarose bead, a cellulose particle, a glass fiber, a controlled pore glass bead and a polystyrene plastic bead. In a related embodiment, the solid phase can be separated from the antibody mixture to remove undesired antibodies that bind to the negative screening protein or peptide.

In another embodiment, the antibody mixture is passed through a column comprising the negative screening protein or peptide affixed to a solid phase to retain an undesired antibody that binds to the negative screening protein or peptide in the column while allowing a desired antibody that does not bind to the negative screening protein or peptide to pass through.

In a further embodiment, a method is provided comprising attaching the produced specific amino acid residue dependent antibody to a surface of a solid phase device suitable for testing for a target protein or peptide. Frequently, a related method is provided that comprises attaching the produced specific amino acid residue dependent antibody to a label.

In a still further embodiment, a specific amino acid residue dependent antibody suitable for testing for a target protein or peptide is provided, which specific amino acid residue dependent antibody is produced by the methods described above.

In another embodiment, a device suitable for testing for a target protein or peptide is provided, which device is produced by the methods described above.

In a further aspect, a method of testing for a target protein or peptide in a sample is provided, which method comprises: contacting a sample containing or suspected of containing a target protein or peptide with a specific amino acid residue dependent antibody under suitable conditions to allow binding of said target protein or peptide, if present in said sample, to said specific amino acid residue dependent antibody, wherein said specific amino acid residue dependent antibody is capable of binding to said target protein or peptide but is incapable of binding to a protein or peptide comprising said target protein or peptide wherein said specific amino acid residue is lacking or unavailable for binding with said antibody; and assessing binding between said target protein or peptide with said specific amino acid residue dependent antibody to determine the presence and/or amount of said target protein or peptide in said sample.

In a related embodiment, the specific amino acid residue dependent antibody is identified by a method comprising: providing an antibody that binds to a target protein or peptide containing a specific amino acid residue; contacting said antibody provided in step a) with a negative screening protein or peptide comprising said target protein or peptide wherein said specific amino acid residue is lacking or unavailable for binding with said antibody; and assessing binding between said antibody and said target protein or peptide and assessing binding between said antibody and said negative screening protein or peptide, whereby identifying an antibody that binds to said target protein or peptide but fails to bind to said negative screening protein or peptide as a specific amino acid residue dependent antibody.

In another embodiment, the specific amino acid residue dependent antibody is produced by a method comprising: providing an antibody mixture wherein at least one of said antibodies in said mixture binds to a target protein or peptide containing a specific amino acid residue; contacting said antibody mixture provided in step a) with a negative screening protein or peptide comprising said target protein or peptide wherein said specific amino acid residue is lacking or unavailable for binding with said antibody; removing from said mixture an undesired antibody that binds to said negative screening protein or peptide; and recovering from said mixture a desired antibody that binds to said target protein or peptide but fails to bind to said negative screening protein or peptide as a specific amino acid residue dependent antibody. Frequently, the sample is a clinical sample, wherein the clinical sample may be a human clinical sample.

In a further embodiment, the binding between the target protein or peptide with the specific amino acid residue dependent antibody is assessed in a homogeneous or a heterogeneous assay format.

In a still further embodiment, the target protein or peptide may be a marker of a biological pathway, a group of cellular structures with identical or similar biological function, a stage of cell cycle, a cell type, a tissue type, an organ type, a developmental stage, a disease or disorder type or stage, or a drug or other treatment. Frequently, the target protein or peptide may be selected from the group consisting of parathyroid hormone (PTH), gastric inhibitory polypeptide (GIP), glucagon-like peptide (GLP), creatine kinase (CK), prostate specific antigen (PSA) and human chorionic gonadotropin (HCG), or a fragment thereof. In a related embodiment, the target protein or peptide is PTH, or a fragment thereof.

In another embodiment, a method is provided further comprising determining and comparing at least two of the parameters selected from the group consisting of the target protein or peptide level, the level of the negative screening protein or peptide level or a protein containing the negative screening protein or peptide, and the sum of the target protein or peptide level and the level of the negative screening protein or peptide level or a protein containing the negative screening protein or peptide. Frequently, the comparison is in the form of a ratio, proportion, difference or product of multiplication. Also, frequently, the target protein is parathyroid hormone (PTH) and the comparison is in the form of a ratio, proportion, difference or product of multiplication between whole PTH and a fragment of PTH.

In a further embodiment, the specific amino acid residue dependent antibody depends on one or more amino acid residues of PTH and the method is used to distinguish the whole PTH (1-84) from a PTH fragment, including, for example, PTH (2-84), PTH (3-84), PTH (4-84), PTH (5-84), PTH (6-84), PTH (7-84), PTH (8-84), PTH (9-84), PTH (10-84), PTH (2-37), PTH (3-37), PTH (4-37), PTH (5-37), PTH (6-37), PTH (7-37), PTH (8-37), PTH (9-37), PTH (10-37), PTH (1-83), PTH (1-82), PTH (1-81), PTH (1-80), PTH (2-83), PTH (2-82), PTH (2-81), PTH (2-80), PTH (3-83), PTH (3-82), PTH (3-81), PTH (3-80), PTH (1-34). Frequently, the specific amino acid residue dependent antibody depends on the first or first two amino acid residues of GIP and/or GLP and the method is used to distinguish among GIP and/or GLP, GIP-1 and/or GLP-1, and GIP-2 and/or GLP-2. On occasion, the specific amino acid residue dependent antibody depends on the amino acid residues of CK located in proximity of CK isoforms (CK-MM and CK—BB and CK-MB) distinction and the method is used to distinguish CK MM from CK BB. Also occasionally, the specific amino acid residue dependent antibody depends on the unique amino acid residues of HCG β subunit and the method is used to distinguish HCG from LH, TSH and FSH.

In a still further embodiment, the method further comprises determining and comparing at least two of the parameters selected from the group consisting of the level of whole PTH (1-84), PTH (2-84), PTH (3-84), PTH (4-84), PTH (5-84), PTH (6-84), PTH (7-84), PTH (8-84), PTH (9-84), PTH (10-84), PTH (1-37), PTH (2-37), PTH (3-37), PTH (4-37), PTH (5-37), PTH (6-37), PTH (7-37), PTH (8-37), PTH (9-37), PTH (10-37), PTH (1-83), PTH (1-82), PTH (1-81), PTH (1-80), PTH (2-83), PTH (2-82), PTH (2-81), PTH (2-80), PTH (3-83), PTH (3-82), PTH (3-81), PTH (3-80), PTH (1-34), PTH (1-35), PTH (1-36) and total PTH level made up of any or all of the above PTH and PTH fragments and unfragmented 1-84 PTH. Frequently, the comparison is in the form of a ratio, proportion, difference or product of multiplication.

In another embodiment, the method is conducted in a format of immuno radio metric assay (IRMA) or acrydinium labeled chemiluminescent assay. Frequently, the method is used for prognosis, diagnosis and/or treatment monitoring of familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, the extent of parathyroid gland surgical removal, oversuppression with vitamin D or a vitamin D analogue or calcium and chronic uremia. On occasion, the hyperparathyroidism is primary hyperparathyroidism caused by primary hyperplasia or adenoma of the parathyroid glands or secondary hyperparathyroidism caused by renal failure.

In a further embodiment, the methods described above are used to distinguish a modified protein or peptide from its naturally occurring counterpart, said modified protein or peptide being different from said naturally occurring counterpart by one or two amino acid residues. Frequently, the modified protein or peptide is selected from the group consisting of insulin, calcitonin, PTH and erythropoietin. Also, on occasion, the present methods are used in a drug discovery process to identify a drug candidate that binds to or masks an epitope of one or two amino acid residues in a target protein or peptide.

In a still further embodiment, methods are provided which are used to distinguish PTH (1-84) from another PTH fragment using a first amino acid residue dependent anti-PTH antibody and a last amino acid residue dependent anti-PTH antibody. Frequently, these methods are used to monitor a PTH fragment that changes with progression of a PTH suppressive therapy and is used to guide an appropriate dosing decision and the PTH suppressive therapy is a vitamin D, calcium or calcimimetic treatment.

In a further aspect, kits are provided for testing for a target protein or peptide, which kit comprises, in a container, a specific amino acid residue dependent antibody suitable for testing for a target protein or peptide produced by the method of claim 1 and an instruction for using said specific amino acid residue dependent antibody in testing for said target protein or peptide. Frequently, such kits further comprises a reagent(s) or means for generating a detectable signal and/or standard curve. In one embodiment, a kit is provided for testing for a target protein or peptide, which kit comprises, in a container, a specific amino acid residue dependent antibody suitable for testing for a target protein or peptide produced by the above methods and instructions for using said specific amino acid residue dependent antibody in testing for said target protein or peptide. These kits frequently further comprise one or more reagent(s) or means for generating a detectable signal and/or standard curve.

### DETAILED DESCRIPTION OF THE INVENTION

For clarity of disclosure, and not by way of limitation, the detailed description of the invention is divided into the subsections that follow.

A. Definitions

Unless defined otherwise, all terms of art, notations and other scientific terms or terminology used herein have the same meaning as is commonly understood by one of ordinary skill in the art to which this invention belongs. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “antibody” is used in the broadest sense. Therefore, an “antibody” can be naturally occurring or man-made such as monoclonal antibodies produced by conventional hybridoma technology. Antibodies of the present invention comprise monoclonal and polyclonal antibodies as well as fragments containing the antigen-binding domain and/or one or more complementarity determining regions of these antibodies.

As used herein, “monoclonal antibody” refers to an antibody obtained from a population of substantially homogeneous antibodies, i.e., the antibodies comprising the population are identical except for possible naturally occurring mutations that are present in minor amounts.

As used herein, “amino acid residue dependent antibody” refers to an antibody that, frequently under physiological conditions, will bind a target, but will not bind the target if the target is missing a component of a specific epitope. Frequently, the component of the epitope that affects binding by a specific antibody comprises one or more amino acid residues such that the antibody will not bind the target if the one or more amino acid residues are not present (or are otherwise unavailable for binding) in the target at their normal position(s). The binding of an “amino acid residue dependent antibody” to a target is not intended to be limited to mere amino acid dependencies as other dependencies are also contemplated.

The term “target” is generally understood to mean any substance desired to be analyzed, either in pure form or in mixture. Frequently, target as used herein refers to a target protein or peptide. On occasion a target may be an oligonucleotide or another moiety described herein.

As used herein, “negative screening protein or peptide” refers to an altered form of a corresponding target protein or peptide, wherein one or more of the amino acid residues that are normally present in the target are lacking or otherwise unavailable in the negative screening protein or peptide for binding by an antibody.

As used herein, “antibody” is used in the broadest sense. Therefore, an “antibody” can be naturally occurring or man-made such as monoclonal antibodies produced by conventional hybridoma technology and/or a functional fragment thereof. Antibodies of the present invention comprise monoclonal and polyclonal antibodies as well as fragments containing the antigen-binding domain and/or one or more complementarity determining regions of these antibodies.

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, the term “specifically binds” refers to the specificity of an antibody such that it preferentially binds to a defined target. Recognition by an antibody of a particular target in the presence of other potential targets is one characteristic of such binding. Specific binding of the presently contemplated antibodies to particular PTH, EPO and other targets is measured through known methods utilizing the tools provided herein.

As used herein, “oligonucleotide” refers to low molecular weight deoxyribo-, ribo-, copolymers of deoxyribo- and ribonucleic acids of chain lengths between 3 and 150. Such oligonucleotides can have modified nucleotide residues such as —O-methoxy, phosphorothio-, methylphosphonates and others known in art.

As used herein, “small molecule” refers to a molecule that, without forming homo-aggregates or without attaching to a macromolecule or adjuvant, is incapable of generating an immune response resulting in an antibody that specifically binds to the small molecule. Preferably, the small molecule has a molecular weight that is about or less than 10,000 Daltons. More preferably, the small molecule has a molecular weight that is about or less than 5,000 Daltons.

As used herein, “inorganic molecule” refers to a molecule that does not contain hydrocarbon group (s).

As used herein, “organic molecule” refers to a molecule that contains hydrocarbon group (s).

As used herein, “biomolecule” refers to an organic compound normally present as an essential component of living organisms.

As used herein, “poly-T tail/spacer” or “poly-T tail” or “poly-T spacer” refers to thymine tails and/or spacers comprising a range of nucleotides represented by Tn nucleotides where “n” may be about 5, 10, 15, 20, 25, etc. In one aspect of the present invention, these spacers may be included as part of the probe nucleotide sequence prior to the 5′ end of the instant probes.

As used herein, “assessing” refers to quantitative and/or qualitative determination of the hybrid formed between the probe and the target nucleotide sequence, e.g., obtaining an absolute value for the amount or concentration of the hybrid, and also of obtaining an index, ratio, percentage, visual or other value indicative of the level of the hybrid. Assessment may be direct or indirect and the chemical species actually detected need not of course be the hybrid itself but may, for example, be a derivative thereof, reduction or disappearance of the probe and/or the target nucleotide sequence, or some further substance.

As used herein, “polynucleotide” refers to a polymeric form of nucleotides of at least 10 bases or base pairs in length, either ribonucleotides or deoxynucleotides or a modified form of either type of nucleotide, and is meant to include single and double stranded forms of DNA and/or RNA. In the art, this term if often used interchangeably with “oligonucleotide”. A polynucleotide can comprise a nucleotide sequence disclosed herein wherein thymidine (T), as shown for example in FIG. 2, can also be uracil (U); this definition pertains to the differences between the chemical structures of DNA and RNA, in particular the observation that one of the four major bases in RNA is uracil (U) instead of thymidine (T).

As used herein, “polypeptide” refers to a polymer of at least about 4, 5, 6, 7, or 8 amino acids. Throughout the specification, standard three letter or single letter designations for amino acids are used. In the art, this term is often used interchangeably with “peptide” or “protein”.

As used herein, “whole parathyroid hormone (PTH)” refers to the complete molecule of PTH or a fragment, derivative or analog thereof that stimulates osteoclasts formation and bone turnover to increase blood calcium levels. For purposes herein, the name “parathyroid hormone (PTH)” is used herein, although all other names are contemplated. See, e.g., Watson et al., MOLECULAR BIOLOGY OF THE GENE, 4th Edition, 1987, The Bejamin/Cummings Pub. Co., p. 224). Whole PTH assay values may be obtained by measuring a sample with a variety of assays.

As used herein, “parathyroid hormone (PTH) fragment” refers to a PTH fragment or derivative that counters the effect of whole PTH or otherwise lacks whole PTH activity in vivo. As also used herein, a PTH fragment may refer to a combination of one or more PTH fragments of variable lengths. See, e.g., Watson, et al. MOLECULAR BIOLOGY OF THE GENE, 4th Edition, 1987, The Bejamin/Cummings Pub. co., p. 224).

As used herein, the terms “total PTH,” “intact PTH” and “total intact PTH” are interchangeable and refer to an assay directed at measuring PTH agonist and PTH antagonist levels.

As used herein, “treatment” means any manner in which the symptoms of a condition, disorder or disease are ameliorated or otherwise beneficially altered. Treatment also encompasses any pharmaceutical use of the compositions herein.

As used herein, “disease or disorder” refers to a pathological condition in an organism resulting from, e.g., infection or genetic defect, and characterized by identifiable symptoms.

As used herein, “adynamic low bone turnover disease” refers to a variety of disorders involving abnormal PTH agonist and/or antagonist levels in a person. This definition is non-limiting in that it does not refer to only one specific disease, it refers to a variety of disorders that may result from abnormal PTH or PTH component levels in a person. As PTH levels are tied to bone turnover rate, abnormally low levels of PTH agonist, abnormally low levels of PTH agonist/antagonist ratios, and abnormally high levels of PTH antagonist may lead to abnormally low bone turnover in a person. In a person, this type of state may indicate the presence of, or susceptibility to, an adynamic low bone turnover disease. Conversely, abnormally high levels of PTH agonist, abnormally high levels of PTH agonist/antagonist ratios, and abnormally low levels of PTH antagonist may lead to abnormally high bone turnover in a person.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

B. Specific Amino Acid Residue Dependant Antibodies

During the search for receptor specific epitopes or epitopes that were not shared with other potential immunoassay cross-reactants it was recognized that, at times, a single amino acid in an epitope was critical for binding. In other words, it was recognized as useful to select for immunoassay antibodies with binding characteristics such that if a particular amino acid was present in its corresponding ligand that there would be binding, and if that amino acid were not present that there would not be binding of the ligand by the antibody.

The present invention encompasses antigens, antibodies and methods of producing antibodies that have a particular specificity to target proteins and/or peptides which contain a specific amino acid residue or multiple amino acid residues, in a series or otherwise. The specific amino acid residue(s) may be located in the N-terminal region of a protein or peptide or in the C-terminal region. Moreover the specific amino acid residue(s) may be located in a region between the N-terminal and C-terminal regions of a protein or peptide, such as the mid-terminal portion. Occasionally, when there is more than one specific amino acid residue, such residues may be dispersed in any one or more of the N-terminal, C-terminal, between these two regions, and/or in all of these regions.

In one embodiment, when the target protein or peptide is a PTH protein or peptide, it may be advantageous to utilize specific amino acid residue dependent antibodies for the N-terminal region of PTH. Frequently, however, when the target protein or peptide is a PTH protein or peptide, it may be advantageous to utilize specific amino acid residue dependent antibodies for the N-terminal and C-terminal regions of the PTH molecule. On occasion, when the target protein or peptide is a PTH protein or peptide, it may be advantageous to utilize specific amino acid residue dependent antibodies for the N-terminal and the mid-terminal and/or C-terminal regions of the PTH molecule.

In another embodiment, when identifying antibodies that recognize a target protein or peptide, it is frequently preferable to employ screening materials having a specific amino acid residue that is lacking or unavailable for binding with the antibody. Such screening materials are useful as a negative screen against antibodies having varying specificities.

Frequently, selected proteins or polypeptides, depending on whether or not they contain a specific amino acid residue (or whether it is otherwise unavailable for binding), can be utilized as positive or negative controls to screen antibodies.

In another embodiment, the target proteins or peptides can be utilized to generate antibodies thereto. Frequently, these antibodies are screened to determined whether they bind to a particular protein or peptide sequence containing a specific amino acid residue.

Frequently, the nature of the modification comprises the removal of one or more specific amino acid residue(s). Generally such residues are removed from a specific epitope or region suspected of containing a particular epitope. Although not intending to be bound by any particular theory, the removal of one or more amino acid residues from an epitope renders the epitope unavailable for binding with a particular amino acid residue dependent antibody as contemplated herein. In one general aspect, the removal of one or more amino acid residues from a particular epitope renders the epitope too small for binding with a particular amino acid residue dependent antibody. Alternatively, the removal of one or more amino acid residues from the epitope affects the primary, secondary, tertiary and/or quaternary structure of the protein such that a particular amino acid residue dependent antibody will not bind with the protein and/or epitope. In one embodiment, an antibody that binds both a negative screening protein that is missing one or more amino acid residues in a particular epitope, and a corresponding target protein, which maintains the one or more amino acid residues in the particular epitope region, is not an amino acid residue dependent antibody as contemplated herein. On occasion, a specific amino acid residue may be modified in such a way to render that residue unavailable for binding. For example, the modification could include phosphorylation, directed mutagenesis, replacement (such as with another amino acid residue, and preferably a non-conservative substitution, but on occasion, a conservative substitution), glycoslylation, among other modes of alteration of replacement.

Upon identification of an antibody that binds to the target protein or peptide, such an antibody can be used to identify further antibodies of interest, depending on whether they compete with the antibody for the target protein or peptide or a particular epitope present on the target protein or peptide.

Target proteins and peptides can be produced by a variety of methods known in the art. For example, such proteins and peptides may be produced by conventional methods including solid-phase peptide synthesis, see, e.g., R. B. Merrifield, et al., Biochemistry 21:5020 (1982), solution phase peptide synthesis or by recombinant technology. Thus, such target peptides or proteins can be isolated or synthetically/recombinantly produced by methods known in the art.

Polyclonal antibodies can be produced in vivo in response to immunization antigens (e.g., proteins, peptides, haptens, chemical compounds, etc.). Anti-serum can be raised in a variety of animals and monitored via an ELISA assay. Often, an antigen comprising a small molecule or a hapten, is coupled to a carrier to induce an immunological reaction. Monoclonal antibodies provide single epitope specificity and a large volume of identical antibody. In contrast, polyclonal antibodies often provide multiple specificities and are occasionally limited in volume, in part, due to the amount of serum that can be obtained from an immunized animal. Nevertheless, the specificity of polyclonal antibodies can be improved by affinity chromatography using purified or synthetic antigen.

Synthetic short peptides are frequently utilized to generate antibodies. This approach involves synthesizing short peptide sequences, often then coupling them to a large carrier molecule, and immunizing the animal of choice with the peptide-carrier molecule.

Although not intending to be bound by any particular theory, the capacity of anti-peptide antibodies to recognize the native protein when utilized in immunoprecipitation or immunohistochemistry staining, depends on the peptide sequence displayed on the surface of the native protein in a conformation similar to that found in the peptide-carrier protein conjugate. Therefore, the successful production of anti-peptide antibodies is often determined by the prediction of the location of certain peptide sequences in the three-dimensional structure of the protein. Protein prediction programs are available for such analysis. Important factors to consider include, for example, protein hydrophilicity, hydropathicity, percent accessible residues, Beta-turn, and flexibility. See Kyte J., Doolittle R. F., 1982. J. Mol. Biol. 157:105-132; Hopp T. P., Woods K. R., 1981. Proc. Natl. Acad. Sci. U.S.A. 78:3824-3828; Janin J., 1979 Nature 277:491-492; Deleage, G., Roux B. 1987 Protein Engineering 1:289-294; and Bhaskaran R., and Ponnuswamy P. K., 1988. Int. J. Pept. Protein Res. 32:242-255. See also the ProtScale website located on the World Wide Web at (.expasy.ch/cgi-bin/protscale.pl) through the ExPasy molecular biology server.

Once produced or obtained, such antigens are useful for generating antibodies thereto using methods known in the art. Frequently, this process involves administering target proteins or peptides to a host animal. Suitable animals include rabbits, mice, sheep, chickens, goats, cows, pigs, rats, etc. A number of other animals may also be suitable for such antibody generation which are readily known and available in the art.

C. Methods for Identifying a Specific Amino Acid Residue Dependent Antibody

In one aspect, the present invention is directed to a method for identifying a specific amino acid residue dependent antibody to a target protein or peptide, which method comprises: providing an antibody that binds to a target protein or peptide containing a specific amino acid residue; contacting said antibody that binds to a target protein or peptide containing a specific amino acid residue with a negative screening protein or peptide comprising said target protein or peptide wherein said specific amino acid residue is lacking or unavailable for binding with said antibody; and assessing binding between said antibody and said target protein or peptide and assessing binding between said antibody and said negative screening protein or peptide, whereby identifying an antibody that binds to said target protein or peptide but fails to bind to said negative screening protein or peptide as a specific amino acid residue dependent antibody.

In one aspect, antibodies are screened to determined whether they bind to a particular peptide sequence. Frequently, this peptide sequence lacks one or more amino acid residues compared with a target protein or peptide. Also frequently, this peptide may include one or more amino acid residues that are unavailable for binding with the antibody. A particular target protein or peptide containing a specific amino acid residue may be used as a positive screen. Also, the target protein or peptide may be included lacking one or more amino acid residues, and is used as a negative screen.

The nature of the target protein or peptide modification is the removal or absence of one or more amino acid residues. Often the negative screening protein or peptide can be synthetically produced without one or more defined amino acid residues. In addition, frequently the native protein or peptide is altered to remove one or more amino acid residues, via enzymatic alteration of otherwise. Also frequently, one or more amino acid residues (or the native protein or peptide, or the synthetically produced protein or peptide) are modified such that they are rendered unavailable for binding with an antibody, via any of a variety of means such as phosphorylation. Also frequently, the negative screening protein or peptide lacks one or more of the amino acid residues of the epitope(s) present in the native protein or peptide, but presents the remaining amino acid residues of that epitope in the appropriate sequence.

Selection is not necessarily an absolute process. If few or no antibodies are found which meet the selection criteria, the criteria may be modified or relaxed. For example, if a panel of several negative screens are used, one might pass an antibody which binds to only one of the negative screens.

Once antibodies are identified that bind to the desired epitope, these antibodies may be used in screening for further antibodies of interest, based on whether they compete with the candidate antibody for a particular epitope.

In another frequent embodiment, screening is performed at a pH consistent with physiological conditions. Specific amino acid dependent antibodies that bind at physiological pH are more likely to be suitable for use in vivo, e.g., in immunotherapy and immunoimaging.

Frequently the amino acid dependent antibody is screened for its ability to bind at a wide range of pH values, both below and above physiological pH. An antibody whose binding is essentially pH-insensitive may be important in an enzyme immunoassay for the antigen.

It is contemplated that the amino acid dependent antibodies identified by the proposed screening method will be useful for immunopurification, immunodiagnosis (including serum or urine diagnosis, histochemical studies, and in vivo immunoimaging), immunotherapy, and other immunological methods.

Frequently, the antibody that binds to a target protein or peptide is provided by immunizing a mammal with an immunizing protein or peptide comprising said target protein or peptide or an immunizing nucleic acid encoding a protein or peptide comprising said target protein or peptide. Also frequently, the immunizing protein or peptide comprises more amino acid residue(s) than the target protein or peptide and the immunizing nucleic acid encodes a protein or peptide that comprises more amino acid residue(s) than the target protein or peptide. Moreover, on occasion, the immunizing protein or peptide is the target protein or peptide and the immunizing nucleic acid encodes the target protein or peptide.

In one embodiment, the antibody that binds to a target protein or peptide is provided by identifying an antibody that is known to bind to the target protein or peptide. This binding is often specific binding. Frequently, the antibody that binds to a target protein or peptide is a polyclonal antibody or polyclonal antiserum. However, often the antibody that binds to a target protein or peptide is a monoclonal antibody or a plurality of monoclonal antibodies.

In another embodiment, the target protein or peptide is a marker of a biological pathway, a group of cellular structures with identical or similar biological function, a stage of cell cycle, a cell type, a tissue type, an organ type, a developmental stage, a disease or disorder type or stage, or a drug or other treatment monitoring. Frequently, the target protein or peptide is a marker of parathyroid gland disease status, renal bone disease, osteoporosis, bone turnover status, the extent of partial or complete parathyroid gland removal. Also frequently, the target protein or peptide is a clinical marker. On occasion, the target protein or peptide is a hormone. Also on occasion, the target protein or peptide comprises a non-proteineous or non-peptidyl moiety. When the target protein or peptide comprises a non-proteineous or non-peptidyl moiety, the non-proteineous or non-peptidyl moiety is frequently selected from the group consisting of an oligonucleotide, a nucleic acid, a vitamin, an oligosaccharide, a carbohydrate, a lipid, a small molecule and a complex or combination thereof. However, on occasion, the target protein or peptide does not comprise a non-proteineous or non-peptidyl moiety.

In one embodiment, the target protein or peptide is selected from the group consisting of parathyroid hormone (PTH), gastric inhibitory polypeptide (GIP), glucagon-like peptide (GLP), creatine kinase (CK), prostate specific antigen (PSA) and human chorionic gonadotropin (HCG), or a fragment thereof.

On occasion, the target protein or peptide is PTH, or a fragment thereof. In this embodiment, the target protein or peptide is frequently used to identify an antibody that depends on the first or first two amino acid residues of PTH. On occasion, the protein or peptide is used to identify an antibody that depends on the last amino acid residue of PTH.

In another embodiment, the negative screening protein or peptide lacks one, two, three, four, five, six, seven, eight, nine, ten or more than ten amino acid residues from the target protein or peptide.

In a frequent embodiment, the identified amino acid residue dependent antibody depends on one specific amino acid residue of the target protein or peptide. In another frequent embodiment, the identified amino acid residue dependent antibody depends on two or more specific amino acid residues of the target protein or peptide.

In one embodiment, the binding (or lack thereof) between the antibody and the negative screening protein or peptide is assessed by a sandwich, competitive or inhibition assay format. Frequently, the binding between the antibody and the negative screening protein or peptide is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, u-capture assay, inhibition assay and avidity assay.

In an occasional embodiment, the provided antibody is contained in an antibody mixture. In this embodiment, frequently the method further comprises recovering the identified amino acid residue dependent antibody from the antibody mixture.

In another embodiment, the present method further comprises attaching the identified specific amino acid residue dependent antibody to a surface of a solid phase device suitable for testing for a target protein or peptide. In a frequent embodiment, the solid phase device is selected from the group consisting of a microtiter plate, a glass slide, a nitrocellulose membrane, a latex bead, a cell, a test tube, a plastic bead, a colloidal gold particle, a colored particle, a magnetic bead and a quantum dot. In another frequent embodiment, the method further comprises attaching the identified specific amino acid residue dependent antibody to a label, and the label is selected from the group consisting of a chemical, an enzymatic, a radioactive, a fluorescent, a fluorescence-quenching, a luminescent and a fluorescence resonance energy transfer (FRET) label.

In another frequent embodiment, a specific amino acid residue dependent antibody suitable for testing for a target protein or peptide is provided, which specific amino acid residue dependent antibody is produced by the present methods. Devices suitable for testing for a target protein or peptide are also contemplated, which devices may be produced by the methods set out herein.

D. Methods for Producing a Specific Amino Acid Residue Dependent Antibody

In another aspect, the present invention is directed to a method for producing a specific amino acid residue dependent antibody to a target protein or peptide, which method comprises: providing an antibody mixture wherein at least one of said antibodies in said mixture binds to a target protein or peptide containing a specific amino acid residue; contacting said antibody mixture with a negative screening protein or peptide comprising said target protein or peptide wherein said specific amino acid residue is lacking or unavailable for binding with said antibody; removing from said mixture an undesired antibody that binds to said negative screening protein or peptide; and recovering from said mixture a desired antibody that binds to said target protein or peptide but fails to bind to said negative screening protein or peptide as a specific amino acid residue dependent antibody.

In one embodiment, the negative screening protein or peptide is attached to a solid phase. Frequently, the solid phase is selected from the group consisting of an agarose bead, a cellulose particle, a glass fiber, a controlled pore glass bead and a polystyrene plastic bead. In another frequent embodiment, the solid phase can be separated from the antibody mixture to remove undesired antibodies that bind to the negative screening protein or peptide.

In another embodiment, the antibody mixture is passed through a column comprising the negative screening protein or peptide affixed to a solid phase to retain an undesired antibody that binds to the negative screening protein or peptide in the column while allowing a desired antibody that does not bind to the negative screening protein or peptide to pass through.

In an occasional embodiment, the present methods further comprise a positive screen to collect the desired antibodies. Such positive screening step may be undertaken before, but is preferably undertaken after binding assessment.

In an occasional embodiment, the present methods further comprise attaching the produced specific amino acid residue dependent antibody to a surface of a solid phase device suitable for testing for a target protein or peptide. Also on occasion, the methods further comprise attaching the produced specific amino acid residue dependent antibody to a label.

In a frequent embodiment, a specific amino acid residue dependent antibody suitable for testing for a target protein or peptide is provided by the present methods.

In another occasional embodiment, a device is provided that is suitable for testing for a target protein or peptide that is produced by the present methods.

E. Methods for Testing for a Target Protein or Peptide in a Sample

In another aspect, the present invention is directed to a method of testing for a target protein or peptide in a sample, which method comprises: contacting a sample containing or suspected of containing a target protein or peptide with a specific amino acid residue dependent antibody under suitable conditions to allow binding of said target protein or peptide, if present in said sample, to said specific amino acid residue dependent antibody, wherein said specific amino acid residue dependent antibody is capable of binding to said target protein or peptide but is incapable of binding to a protein or peptide comprising said target protein or peptide wherein said specific amino acid residue is lacking or unavailable for binding with said antibody; and assessing binding between said target protein or peptide with said specific amino acid residue dependent antibody to determine the presence and/or amount of said target protein or peptide in said sample.

In another aspect, the present invention is directed to the above method, wherein the specific amino acid residue dependent antibody is identified by a method comprising: providing an antibody that binds to a target protein or peptide containing a specific amino acid residue; contacting said antibody with a negative screening protein or peptide comprising said target protein or peptide wherein said specific amino acid residue is lacking or unavailable for binding with said antibody; and assessing binding between said antibody and said target protein or peptide and assessing binding between said antibody and said negative screening protein or peptide, whereby identifying an antibody that binds to said target protein or peptide but fails to bind to said negative screening protein or peptide as a specific amino acid residue dependent antibody.

In another aspect, the present invention is directed to the above method(s), wherein the specific amino acid residue dependent antibody is produced by a method comprising: providing an antibody mixture wherein at least one of said antibodies in said mixture binds to a target protein or peptide containing a specific amino acid residue; contacting said antibody mixture with a negative screening protein or peptide comprising said target protein or peptide wherein said specific amino acid residue is lacking or unavailable for binding with said antibody; removing from said mixture an undesired antibody that binds to said negative screening protein or peptide; and recovering from said mixture a desired antibody that binds to said target protein or peptide but fails to bind to said negative screening protein or peptide as a specific amino acid residue dependent antibody.

In a frequent embodiment, the sample is a clinical sample. Also frequently, the clinical sample is a human clinical sample.

In another embodiment, the present methods further comprise attaching the specific amino acid residue dependent antibody to a surface of a device suitable for testing for a target protein or peptide before contacting the specific amino acid residue dependent antibody with the sample. Frequently, however, the present methods further comprise attaching the identified specific amino acid residue dependent antibody to a label.

In a frequent embodiment, the binding between the target protein or peptide with the specific amino acid dependent antibody is assessed by a sandwich or competitive assay format. Frequently, the binding between the target protein or peptide with the specific amino acid residue dependent antibody is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, u-capture assay, inhibition assay and avidity assay.

Also frequently, the specific amino acid residue dependent antibody binds to the target protein or peptide specifically.

In one embodiment, the target protein or peptide is a marker of a biological pathway, a group of cellular structures with identical or similar biological function, a stage of cell cycle, a cell type, a tissue type, an organ type, a developmental stage, a disease or disorder type or stage, or a drug or other treatment. Frequently, the target protein or peptide is a clinical marker. Often, the target protein or peptide is a marker of parathyroid gland disease status, renal bone disease, osteoporosis, bone turnover status, the extent of partial or complete parathyroid gland removal. On occasion, the target protein or peptide is a hormone or a non-proteineous or non-peptidyl moiety such as an oligonucleotide, a nucleic acid, a vitamin, an oligosaccharide, a carbohydrate, a lipid, a small molecule and a complex or combination thereof. Also on occasion, the target protein or peptide is selected from the group consisting of parathyroid hormone (PTH), gastric inhibitory polypeptide (GIP), glucagon-like peptide (GLP), creatine kinase (CK), prostate specific antigen (PSA) and human chorionic gonadotropin (HCG), or a fragment thereof

In another embodiment, the present method further comprises determining and comparing at least two of the parameters selected from the group consisting of the target protein or peptide level, the level of the negative screening protein or peptide level or a protein containing the negative screening protein or peptide, and the sum of the target protein or peptide level and the level of the negative screening protein or peptide level or a protein containing the negative screening protein or peptide. Often, the comparison is in the form of a ratio, proportion, difference or product of multiplication. On occasion, the target protein is parathyroid hormone (PTH) and the comparison is in the form of a ratio, proportion, difference or product of multiplication between unfragmented PTH and a fragment of PTH.

When the target protein is PTH, frequently the specific amino acid residue dependent antibody depends on one or more of the amino acid residues of PTH and the method is used to distinguish the whole PTH (1-84) from PTH (2-84), PTH (3-84), PTH (4-84), PTH (5-84), PTH (6-84), PTH (7-84), PTH (8-84), PTH (9-84), PTH (10-84), PTH (2-37), PTH (3-37), PTH (4-37), PTH (5-37), PTH (6-37), PTH (7-37), PTH (8-37), PTH (9-37), PTH (10-37), PTH (1-83), PTH (1-82), PTH (1-81), PTH (1-80), PTH (2-83), PTH (2-82), PTH (2-81), PTH (2-80), PTH (3-83), PTH (3-82), PTH (3-81), PTH (3-80), PTH (1-34). Also frequently, the method further comprises determining and comparing at least two of the parameters selected from the group consisting of the level of whole PTH (1-84), PTH (2-84), PTH (3-84), PTH (4-84), PTH (5-84), PTH (6-84), PTH (7-84), PTH (8-84), PTH (9-84), PTH (10-84), PTH (1-37), PTH (2-37), PTH (3-37), PTH (4-37), PTH (5-37), PTH (6-37), PTH (7-37), PTH (8-37), PTH (9-37), PTH (10-37), PTH (1-83), PTH (1-82), PTH (1-81), PTH (1-80), PTH (2-83), PTH (2-82), PTH (2-81), PTH (2-80), PTH (3-83), PTH (3-82), PTH (3-81), PTH (3-80), PTH (1-34), PTH (1-35), PTH (1-36) and total PTH level made up of any or all of the above PTH and PTH fragments and unfragmented 1-84 PTH. Frequently, the comparison is in the form of a ratio, proportion, difference or product of multiplication.

In a frequent embodiment, the present methods are conducted in a format of immuno radiometric assay (IRMA) or acrydinium labeled chemiluminescent assay. Also frequently, the present method is used for prognosis, diagnosis and/or treatment monitoring of familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, the extent of parathyroid gland surgical removal, oversuppression with vitamin D or a vitamin D analogue or calcium and chronic uremia. Often, the hyperparathyroidism is primary hyperparathyroidism caused by primary hyperplasia or adenoma of the parathyroid glands or secondary hyperparathyroidism caused by renal failure.

In an occasional embodiment, the specific amino acid residue dependent antibody depends on the first or first two amino acid residues of GIP and/or GLP and the method is used to distinguish among GIP and/or GLP, GIP-1 and/or GLP-1, and GIP-2 and/or GLP-2. Frequently, the specific amino acid residue dependent antibody depends on the amino acid residues of CK located in proximity of CK isoforms (CK-MM and CK—BB and CK-MB) distinction and the method is used to distinguish CK MM from CK BB. Also frequently, the specific amino acid residue dependent antibody depends on the unique amino acid residues of HCG β subunit and the method is used to distinguish HCG from LH, TSH and FSH.

In one embodiment, the present methods are used to distinguish a modified protein or peptide from its naturally occurring counterpart, said modified protein or peptide being different from said naturally occurring counterpart by one or two amino acid residues. Frequently, the modified protein or peptide is selected from the group consisting of insulin, calcitonin, PTH and erythropoietin. On occasion, the present methods are conducted as a part of clinical trial.

In an occasional embodiment, the methods are used in a drug discovery process to identify a drug candidate that binds to or masks an epitope of one or two amino acid residues in a target protein or peptide. Also on occasion, the methods are used to distinguish PTH (1-84) from another PTH fragment using a first amino acid residue dependent anti-PTH antibody and a last amino acid residue dependent anti-PTH antibody. In an occasion embodiment, the methods are used to monitor a PTH fragment that changes with progression of a PTH suppressive therapy and is used to guide an appropriate dosing decision. Frequently, the PTH suppressive therapy is a vitamin D, calcium or calcimimetic treatment.

F. Kits

In a frequent embodiment, a kit is provided for testing for a target protein or peptide, which kit comprises, in a container, a specific amino acid residue dependent antibody suitable for testing for a target protein or peptide produced by the above methods and instructions for using said specific amino acid residue dependent antibody in testing for said target protein or peptide. Frequently, the kit further comprises a reagent(s) or means for generating a detectable signal and/or standard curve.

In another embodiment, a kit is provided for testing for a target protein or peptide, which kit comprises, in a container, a specific amino acid residue dependent antibody suitable for testing for a target protein or peptide produced by the method of claim 34 and an instruction for using said specific amino acid residue dependent antibody in testing for said target protein or peptide. Frequently, the kit further comprises a reagent(s) or means for generating a detectable signal and/or standard curve.

The invention also provides for kits for carrying out the methods of the invention. Such kits comprise in one or more containers a means for determining and monitoring the level of parathyroid hormone (PTH) agonist in a renal patient having secondary hyperparathyroidism; in one or more containers, a means for determining and monitoring the level of PTH antagonist in the patient alone or in combination with other agents; and a means for administering a therapeutic to the patient that suppresses PTH agonist whereby the amount of therapeutic administered is adjusted such that PTH agonist levels are decreased and the level of PTH antagonist is minimized. Examples of means for determining and monitoring PTH agonist levels in a patent comprise a variety of PTH assays further described herein. And, examples of means for determining and monitoring PTH antagonist levels in a patent comprise a variety of PTH assays further described herein. Preferred therapeutic forms would be in combination with sterile saline, dextrose solution, or buffered solution, or other pharmaceutically acceptable sterile fluid. Alternatively, the therapeutic composition may be lyophilized or desiccated; in this instance, the kit optionally further comprises in a container a pharmaceutically acceptable solution, preferably sterile, to reconstitute the complex to form a solution for injection purposes. Exemplary pharmaceutically acceptable solutions are saline and dextrose solution.

In one aspect, a kit of the invention further comprises a needle or syringe as a means for administering a therapeutic to a patient, preferably packaged in sterile form, for injecting the composition, and/or a packaged alcohol pad. Instructions are optionally included for administration of composition by a physician or by the patient.

Other features and advantages of the invention will be apparent from the following detailed description, and from the claims.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLES

### Example 1

Human calcitonin (1-32), P. Sieber et al., Helv. Chim. Acta 51:2057 (1968), K. H. Antonin et al, Clin. Sci. 83:627 (1992), is obtained or synthesized comprising an amino acid sequence:

CGNLSTCMLG TYTQDFNKFH TFPQTAIGVG AP

Amino terminal calcitonin protein fragments are synthesized comprising:

| | |
|---|---|
| CGNLSTCMLGTYTQ | Calcitonin (1-14) |
| CGNLSTCMLGTYTQD | Calcitonin (1-15) |

A carboxyl terminal calcitonin protein fragment is synthesized comprising:

| | |
|---|---|
| NKFHTFPQTAIGVGAP | Calcitonin (17-32) |

The amino terminals of the calcitonin (1-14) and (1-15) fragments and the whole calcitonin protein (1-32) are then attached to one or more affinity purification columns. The carboxyl terminal of the calcitonin (17-32) fragment is also attached to an affinity purification column. The attachment may be effected through means known in the art, such as a through the use of a linker at the amino or carboxyl terminal ends of the calcitonin protein and protein fragments.

One or more goats are immunized with whole calcitonin protein (1-32). Alternatively, one or more goats are immunized with calcitonin (1-14) and/or calcitonin (17-32). Thereafter, serum from the immunized goat(s) is/are pooled and affinity purified against whole length calcitonin (1-32). After affinity purification against calcitonin (1-32), the reaction product of that affinity purification is negatively absorbed with calcitonin (1-14) and calcitonin (17-32). Thereafter, the unbound antibody present in the reaction product after the negative absorption is again affinity purified against calcitonin (1-32) to determine whether any antibodies present will bind with the whole length calcitonin (1-32). Antibodies which thereafter bind with calcitonin (1-32) are determined to be specific for at least amino acid residues 15 and/or 16 of the calcitonin protein molecule.

Goat serum is again pooled and affinity purified against whole length calcitonin (1-32). After affinity purification against calcitonin (1-32), the reaction product of that affinity purification is negatively absorbed with calcitonin (1-15) and calcitonin (17-32). Thereafter, the unbound antibody present in the reaction product after the negative absorption is again affinity purified against calcitonin (1-32) to determine whether any antibodies present will bind with the whole length calcitonin (1-32). Antibodies which thereafter bind with calcitonin (1-32) are determined to be specific for at least amino acid residue 16 of the calcitonin protein molecule.

The foregoing represents specific conditions that can be adjusted to identify amino acid residue dependant antibodies whose binding is dependant on amino acid residues other than (or inclusive of) 15 and/or 16 of the calcitonin molecule. Given the description provided, one of skill in the art would understand the required modifications to the above methods to determine other antibodies whose binding depends from other amino acid residues (or combinations thereof) in the calcitonin protein molecule, that do not depart from the gist and scope of the present invention.

### Example 2

Human PTH (1-84) is obtained or synthesized comprising an amino acid sequence:

SVSEIQLMHN LGKHLNSMER VEWLRKKLQD VHNFVALGAP
LAPRDAGSQR PRKKEDNVLV ESHEKSLGEA NKADVNVLTK
AKSQ

Human PTH (1-34) is obtained comprising an amino acid sequence:

SVSEIQLMHN LGKHLNSMER VEWLRKKLQD VHNF

PTH protein fragments are synthesized comprising:

| | |
|---|---|
| VSEIQLMHN LGKHLNSMER VEWLRKKLQD VHNF | PTH (2-34) |
| SEIQLMHN LGKHLNSMER VEWLRKKLQD VHNF | PTH (3-34) |
| EKSLGEA NKADVNVLTK AKSQ | PTH (64-84) |
| EKSLGEA NKADVNVLTK AKS | PTH (64-83) |
| EKSLGEA NKADVNVLTK AK | PTH (64-82) |

First Amino Acid Dependant Antibody for PTH

The carboxyl terminal of the PTH (2-34) fragment and the PTH (1-34) are then attached to one or more affinity purification columns. The attachment may be effected through means known in the art, such as a through the use of a linker at the carboxyl terminal end of the PTH fragments.

One or more goats are immunized with PTH (1-34). Alternatively, one or more goats are immunized with PTH (2-34). Thereafter, serum from the immunized goat(s) is/are pooled and affinity purified against PTH (1-34). After affinity purification against PTH (1-34), the reaction product of that affinity purification is negatively absorbed with PTH (2-34). Thereafter, the unbound antibody present in the reaction product after the negative absorption is again affinity purified against PTH (1-34) to determine whether any antibodies present will bind with PTH (1-34). Antibodies which thereafter bind with PTH (1-34) are determined to be specific for at least amino acid residue 1 of the PTH (1-34) molecule.

First and/or Second Amino Acid Dependant Antibody for PTH

The carboxyl terminal of the PTH (3-34) fragment and the PTH (1-34) are attached to one or more affinity purification columns. The attachment may be effected through means known in the art, such as a through the use of a linker at the carboxyl terminal end of the PTH fragments.

One or more goats are immunized with PTH (1-34). Alternatively, one or more goats are immunized with PTH (3-34). Thereafter, serum from the immunized goat(s) is/are pooled and affinity purified against PTH (1-34). After affinity purification against PTH (1-34), the reaction product of that affinity purification is negatively absorbed with PTH (3-34). Thereafter, the unbound antibody present in the reaction product after the negative absorption is again affinity purified against PTH (1-34) to determine whether any antibodies present will bind with PTH (1-34). Antibodies which thereafter bind with PTH (1-34) are determined to be specific for at least amino acid residues 1 and/or 2 of the PTH (1-34) molecule.

Last Amino Acid Dependant Antibody for PTH

The amino terminal of the PTH (64-84) fragment and the PTH (64-83) are attached to one or more affinity purification columns. The attachment may be effected through means known in the art, such as a through the use of a linker at the amino terminal end of the PTH fragments.

One or more goats are immunized with PTH (1-84). Alternatively, one or more goats are immunized with PTH (64-84), PTH (64-83) and/or another PTH C-terminal fragment. Thereafter, serum from the immunized goat(s) is/are pooled and affinity purified against PTH (64-84). After affinity purification against PTH (64-84), the reaction product of that affinity purification is negatively absorbed with PTH (64-83). Thereafter, the unbound antibody present in the reaction product after the negative absorption is again affinity purified against PTH (64-84) to determine whether any antibodies present will bind with PTH (64-84). Antibodies which thereafter bind with PTH (64-84) are determined to be specific for at least amino acid residue 84 the PTH (64-84) molecule.

Last and/or Second to Last Amino Acid Dependant Antibody for PTH

The amino terminal of the PTH (64-84) fragment and the PTH (64-82) are attached to one or more affinity purification columns. The attachment may be effected through means known in the art, such as a through the use of a linker at the amino terminal end of the PTH fragments.

One or more goats are immunized with PTH (1-84). Alternatively, one or more goats are immunized with PTH (64-84), PTH (64-83), PTH (64-82) and/or another PTH C-terminal fragment. Thereafter, serum from the immunized goat(s) is/are pooled and affinity purified against PTH (64-84). After affinity purification against PTH (64-84), the reaction product of that affinity purification is negatively absorbed with PTH (64-82). Thereafter, the unbound antibody present in the reaction product after the negative absorption is again affinity purified against PTH (64-84) to determine whether any antibodies present will bind with PTH (64-84). Antibodies which thereafter bind with PTH (64-84) are determined to be specific for at least amino acid residues 83 and/or 84 of the PTH (64-84) molecule.

The above examples are representative of the larger methodology provided herein and are thus non-limiting. Alternative (or other) immunization and/or purification schemes are contemplated utilizing the same or different experimental components. The foregoing represents specific conditions that can be adjusted to identify amino acid residue dependant antibodies whose binding is dependant on amino acid residues other than (or inclusive of) 1, 2, 83 and/or 84 of the PTH protein molecule. Given the description provided, one of skill in the art would understand the required modifications to the above methods to determine other antibodies whose binding depends from other amino acid residues (or combinations thereof) in the PTH protein molecule, that do not depart from the gist and scope of the present invention. Moreover, the present methods are useful to identify specific amino acid residue dependent antibodies specific for a variety of other proteins or antigens described or contemplated herein, given knowledge or determination of the protein or peptide sequence.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A method for identifying a specific amino acid residue dependent antibody to a target protein or peptide, which method comprises:
a) providing an antibody that binds to a target protein or peptide containing a specific amino acid residue;
b) contacting said antibody provided in step a) with a negative screening protein or peptide comprising said target protein or peptide wherein said specific amino acid residue is lacking or unavailable for binding with said antibody; and
c) assessing binding between said antibody and said target protein or peptide and assessing binding between said antibody and said negative screening protein or peptide, whereby identifying an antibody that binds to said target protein or peptide, but fails to bind to said negative screening protein or peptide, as a specific amino acid residue dependent antibody.

2. The method of claim 1, wherein the antibody that binds to a target protein or peptide is provided by immunizing a mammal with an immunizing protein or peptide comprising said target protein or peptide or an immunizing nucleic acid encoding a protein or peptide comprising said target protein or peptide.

3. The method of claim 2, wherein the immunizing protein or peptide comprises more amino acid residue(s) than the target protein or peptide and the immunizing nucleic acid encodes a protein or peptide that comprises more amino acid residue(s) than the target protein or peptide.

4. The method of claim 2, wherein the immunizing protein or peptide is the target protein or peptide and the immunizing nucleic acid encodes the target protein or peptide.

5. The method of claim 1, wherein the antibody that binds to a target protein or peptide is provided by identifying an antibody that is known to bind to the target protein or peptide.

6. The method of claim 1, wherein the antibody binds to a target protein or peptide specifically.

7. The method of claim 1, wherein the target protein or peptide is a marker of parathyroid gland disease status, renal bone disease, osteoporosis, bone turnover status, the extent of partial or complete parathyroid gland removal.

8. The method of claim 1, wherein the target protein or peptide is PTH, or a fragment thereof.

9. The method of claim 1, wherein the negative screening protein or peptide lacks one, two, three, four, five, six, seven, eight, nine, ten or more than ten amino acid residues from the target protein or peptide.

10. The method of claim 1, wherein the identified amino acid residue dependent antibody depends on one specific amino acid residue of the target protein or peptide.

11. The method of claim 1, wherein the identified amino acid residue dependent antibody depends on two or more specific amino acid residues of the target protein or peptide.

12. A specific amino acid residue dependent antibody, which specific amino acid residue dependent antibody is produced by the method of claim 2.

13. The method of claim 1, further comprising attaching the identified specific amino acid residue dependent antibody to a surface of a solid phase device suitable for testing for a target protein or peptide.

14. The method of claim 1, further comprising attaching the identified specific amino acid residue dependent antibody to a label.

15. A specific amino acid residue dependent antibody suitable for testing for a target protein or peptide, which specific amino acid residue dependent antibody is produced by the method of claim 1.

16. A device suitable for testing for a target protein or peptide, which device is produced by the method of claim 13.

17. A method for producing a specific amino acid residue dependent antibody to a target protein or peptide, which method comprises:
a) providing an antibody mixture wherein at least one of said antibodies in said mixture binds to a target protein or peptide containing a specific amino acid residue;
b) contacting said antibody mixture provided in step a) with a negative screening protein or peptide comprising said target protein or peptide wherein said specific amino acid residue is lacking or unavailable for binding with said antibody;
c) removing from said mixture an undesired antibody that binds to said negative screening protein or peptide; and
d) recovering from said mixture a desired antibody that binds to said target protein or peptide but fails to bind to said negative screening protein or peptide as a specific amino acid residue dependent antibody.

18. The method of claim 17, wherein the negative screening protein or peptide is attached to a solid phase.

19. The method of claim 18, wherein the solid phase can be separated from the antibody mixture to remove undesired antibodies that bind to the negative screening protein or peptide.

20. The method of claim 17, wherein the antibody mixture is passed through a column comprising the negative screening protein or peptide affixed to a solid phase to retain an undesired antibody that binds to the negative screening protein or peptide in the column while allowing a desired antibody that does not bind to the negative screening protein or peptide to pass through.

21. The method of claim 17, further comprising a positive screen to collect the desired antibodies.

22. The method of claim 17, further comprising attaching the produced specific amino acid residue dependent antibody to a surface of a solid phase device suitable for testing for a target protein or peptide.

23. The method of claim 17, further comprising attaching the produced specific amino acid residue dependent antibody to a label.

24. A specific amino acid residue dependent antibody suitable for testing for a target protein or peptide, which specific amino acid residue dependent antibody is produced by the method of claim 17.

25. A device suitable for testing for a target protein or peptide, which device is produced by the method of claim 22.

26. A method of testing for a target protein or peptide in a sample, which method comprises:
a) contacting a sample containing or suspected of containing a target protein or peptide with a specific amino acid residue dependent antibody under suitable conditions to allow binding of said target protein or peptide, if present in said sample, to said specific amino acid residue dependent antibody, wherein said specific amino acid residue dependent antibody is capable of binding to said target protein or peptide but is incapable of binding to a protein or peptide comprising said target protein or peptide wherein said specific amino acid residue is lacking or unavailable for binding with said antibody; and
b) assessing binding between said target protein or peptide with said specific amino acid residue dependent antibody to determine the presence and/or amount of said target protein or peptide in said sample.

27. The method of claim 26, wherein the specific amino acid residue dependent antibody is identified by a method comprising:
a) providing an antibody that binds to a target protein or peptide containing a specific amino acid residue;
b) contacting said antibody provided in step a) with a negative screening protein or peptide comprising said target protein or peptide wherein said specific amino acid residue is lacking or unavailable for binding with said antibody; and
c) assessing binding between said antibody and said target protein or peptide and assessing binding between said antibody and said negative screening protein or peptide, whereby identifying an antibody that binds to said target protein or peptide but fails to bind to said negative screening protein or peptide as a specific amino acid residue dependent antibody.

28. The method of claim 26, wherein the specific amino acid residue dependent antibody is produced by a method comprising:
a) providing an antibody mixture wherein at least one of said antibodies in said mixture binds to a target protein or peptide containing a specific amino acid residue;
b) contacting said antibody mixture provided in step a) with a negative screening protein or peptide comprising said target protein or peptide wherein said specific amino acid residue is lacking or unavailable for binding with said antibody;
c) removing from said mixture an undesired antibody that binds to said negative screening protein or peptide; and
d) recovering from said mixture a desired antibody that binds to said target protein or peptide but fails to bind to said negative screening protein or peptide as a specific amino acid residue dependent antibody.

29. The method of claim 26, further comprising attaching the specific amino acid residue dependent antibody to a surface of a device suitable for testing for a target protein or peptide before contacting the specific amino acid residue dependent antibody with the sample.

30. The method of claim 26, further comprising attaching the identified specific amino acid residue dependent antibody to a label.

31. The method of claim 26, wherein the specific amino acid residue dependent antibody binds to the target protein or peptide specifically.

32. The method of claim 26, wherein the target protein or peptide is a marker of parathyroid gland disease status, renal bone disease, osteoporosis, bone turnover status, the extent of partial or complete parathyroid gland removal.

33. The method of claim 26, wherein the target protein or peptide is a clinical marker.

34. The method of claim 26, further comprising determining and comparing at least two of the parameters selected from the group consisting of the target protein or peptide level, the level of the negative screening protein or peptide level or a protein containing the negative screening protein or peptide, and the sum of the target protein or peptide level and the level of the negative screening protein or peptide level or a protein containing the negative screening protein or peptide.

35. The method of claim 34, wherein the comparison is in the form of a ratio, proportion, difference or product of multiplication.

36. The method of claim 34, wherein the target protein is parathyroid hormone (PTH) and the comparison is in the form of a ratio, proportion, difference or product of multiplication between unfragmented PTH and a fragment of PTH.

37. The method of claim 26, wherein the target protein or peptide is selected from the group consisting of parathyroid hormone (PTH), gastric inhibitory polypeptide (GIP), glucagon-like peptide (GLP), creatine kinase (CK), prostate specific antigen (PSA) and human chorionic gonadotropin (HCG), or a fragment thereof.

38. The method of claim 36, which is used for prognosis, diagnosis and/or treatment monitoring of familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, the extent of parathyroid gland surgical removal, oversuppression with vitamin D or a vitamin D analogue or calcium and chronic uremia.

39. The method of claim 26, wherein the specific amino acid residue dependent antibody depends on the first or first two amino acid residues of GIP and/or GLP and the method is used to distinguish among GIP and/or GLP, GIP-1 and/or GLP-1, and GIP-2 and/or GLP-2.

40. The method of claim 26, wherein the specific amino acid residue dependent antibody depends on the amino acid residues of CK located in proximity of CK isoforms (CK-MM and CK—BB and CK-MB) distinction and the method is used to distinguish CK MM from CK BB.

41. The method of claim 26, wherein the specific amino acid residue dependent antibody depends on the unique amino acid residues of HCG β subunit and the method is used to distinguish HCG from LH, TSH and FSH.

42. The method of claim 26, which is used to distinguish a modified protein or peptide from its naturally occurring counterpart, said modified protein or peptide being different from said naturally occurring counterpart by one or two amino acid residues.

43. The method of claim 42, wherein the modified protein or peptide is selected from the group consisting of insulin, calcitonin, PTH and erythropoietin.

44. A kit for testing for a target protein or peptide, which kit comprises, in a container, a specific amino acid residue dependent antibody suitable for testing for a target protein or peptide produced by the method of claim 1 and an instruction for using said specific amino acid residue dependent antibody in testing for said target protein or peptide.

45. The kit of claim 44, which further comprises a reagent(s) or means for generating a detectable signal and/or standard curve.

46. A kit for testing for a target protein or peptide, which kit comprises, in a container, a specific amino acid residue dependent antibody suitable for testing for a target protein or peptide produced by the method of claim 17 and an instruction for using said specific amino acid residue dependent antibody in testing for said target protein or peptide.

47. The kit of claim 46, which further comprises a reagent(s) or means for generating a detectable signal and/or standard curve.
